(19) United States
(12) Patent Application Publication
(10) Pub. No.: US 2010/0234718 A1
(43) Pub. Date: Sep. 16, 2010

(54) OPEN ARCHITECTURE MEDICAL COMMUNICATION SYSTEM

(76) Inventors: Anand Sampath, Corona, CA (US); Ammar Al-Ali, Tustin, CA (US); Eric Karl Kinast, Santa Ana, CA (US); Massi Joe E. Kiani, Laguna Niguel, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/723,526

(22) Filed: Mar. 12, 2010

Related U.S. Application Data
(60) Provisional application No. 61/159,764, filed on Mar. 12, 2009.

(51) Int. Cl. A61B 6/00

(52) U.S. Cl. 600/407

(57) ABSTRACT

The present disclosure provides an open architecture communication system for patient monitoring devices and other patient care equipment. The open architecture of the present system allows devices running different operating systems and software to communicate correctly and efficiently with the care center network. One aspect of the system includes a patient monitoring virtual machine which allows patient monitoring devices and other patient care equipment to allow for communications with other devices on the network or other networks and automatic configuration of devices. Aspects of the system also provide for relocation of post-processing functions, arrangement of devices into domains, and/or management of devices using servers and other management systems.

### CROSS-REFERENCE TO RELATED APPLICATIONS

This application claims the benefit of U.S. Provisional Patent Application No. 61/159,764, filed Mar. 12, 2009, which is incorporated in its entirety herein by reference. The present application is also related to U.S. patent application Ser. No. 11/633,656, entitled “Physiological Alarm Notification System,” filed Dec. 4, 2006, incorporated in its entirety herein by reference.

### FIELD OF THE INVENTION

This invention relates to a system, apparatus, and method for providing communications for patient care equipment. In certain embodiments, the invention relates to providing an open architecture communication system.

### BACKGROUND

Hospitals, nursing homes, and other patient care facilities typically include patient monitoring devices and other patient care equipment at one or more bedsides in the facility. Patient monitoring devices, for example, generally include sensors, processing equipment, and displays for obtaining and analyzing a medical patient's physiological parameters. Physiological parameters include, for example, respiratory rate, blood gas levels, pulse, ECG, EEG, glucose and blood pressure, among others. Clinicians, including doctors, nurses, and certain other medical personnel use the physiological parameters obtained from the medical patient to diagnose illnesses and to prescribe treatments. Clinicians also use the physiological parameters to monitor a patient during various clinical situations to determine whether to increase the level of medical care given to the patient. Other patient care equipment can also used to assist in the care of the patient including medicine dispensing equipment, communication equipment, alarm signals and other devices.

Various proprietary networks have been used in hospitals to aid clinicians in receiving information from medical equipment during normal operation. One technique for transmitting physiological data throughout a hospital is to include a server or workstation at one or more central nurses' stations in the hospital. Physiological information from several patients may be observed at the server or workstation. However, this conventional central station paradigm does not adequately address the workflow models in hospital general care floors where nurse to patient ratios are often 1:6 or greater, where nurses have a lower skill set than ICUs, and where patients are often housed in private or semi-private rooms not in direct view of clinicians. Some systems are adapted to include clinician paging to enable secondary alarm notification to mobile health care personnel, but such systems still rely on a central station concept and therefore bear the cost for such components.

Many hospitals have their own unique proprietary network infrastructure. These networks generally include proprietary connectors, communications hardware, servers, and software. Both wired and wireless proprietary networks are used. For example, patients who are bed-ridden may be connected to a bedside device that is wired to the network. Ambulatory patients may wear a mobile monitoring device that uses radio frequency signals and telemetry techniques to transmit physiological information over the network.

There are also drawbacks to using proprietary networks. One drawback is that proprietary networks tend to be costly to obtain, setup, and maintain. Patient monitoring devices must be designed to interlace with the proprietary network or special adaptors must be used. Specialized servers and server software must be obtained, and extensive training may be required to teach clinicians how to use unfamiliar interfaces. In addition, updating aging proprietary networks with newer, more standardized technology may require the design of new adaptors, software, and additional training. Another drawback is that proprietary networks may provide only limited amounts of data to remote devices due to physical limitations in legacy hardware and software.

Patient monitoring devices used in some proprietary networks include communications hardware within the device. Consequently, replacing or upgrading these patient monitoring devices simultaneously requires replacement of the communications hardware, which is cost-inefficient. In other systems, patient monitoring devices instead connect to a docking station that contains communications hardware. However, these docking stations are often wired into a proprietary network and suffer the drawbacks attendant to such networks.

Additionally, patient monitoring devices must be highly robust and able to tolerate component and device failures. Robustness is of particular importance where devices are used to monitor patient status in health care facilities. For example, if a component of a monitor fails, such as an alarm, the alarm conditions may go unnoticed. In some situations, if a patient monitoring device experiences a failure during operation, the failure may necessitate disabling the entire device. For example, if the device is operating off of battery power and the battery power level runs low, the device will be forced to shut down, thus disrupting the monitoring of the patient and requiring the use of a back up monitor.

### SUMMARY

The present disclosure provides an open architecture communication system for patient monitoring devices and other patient care equipment. The open architecture system of the present system allows devices running different operating systems and software to communicate correctly and efficiently with each other and other devices on the care center network. One aspect of the system includes a patient monitoring virtual machine which allows patient monitoring devices and other patient care equipment to communicate with each other in an open architecture system that provides for sharing and reallocation of resources, including instructions, alarms, measurements, configuration information, or the like, with other devices on the network, such as, for example, other patient monitors of the same or different type, central monitoring stations, pagers, cell phones, remote monitoring stations, non-central local monitoring stations, other patient care equipment, etc.

In an embodiment, aspects of the communications are abstracted by the virtual machine to provide a system configured to allow communications between multiple different platforms without differences in the effect of the communications between platforms. The virtual machine can operate on the patient care device, such as the patient monitor. Alternatively, the virtual machine can operate on separate network device or on the end user device. The virtual machine manages various aspects of the communications between the patient care device and other care facility devices. Other embodiments can be used without a virtual machine implementation. Rather, the communication operations can be implemented by the main software running on each monitor or other networked device. In an embodiment, some monitors and networked devices operate using a virtual machine while others do not use a virtual machine.

In an embodiment, some functions and communications of the open architecture system are protected. For example, in an embodiment, certain spaces are partitioned to protect sensitive or high priority communications or instructions. In an embodiment, manufacturer, measurement and/or connectivity spaces are partitioned.

In an embodiment, measurement devices are provided with a system for synchronizing clocks such that the measurement obtained from each device can be synchronized on the end user device or synchronized for further processing.

In an embodiment, the system is configured to allow advertisements, messages (including audio, video and text), or other audio or visual communications to be displayed or played on the measurement device. In an embodiment, the audio or visual communications are only played at certain low priority points while measurements are taken.

In an embodiment, the system is configured to relocate post-processing functions of one patient care device to another patient care device. The relocation may be controlled by rules operating on a patient care device or on a server connected via a network.

In an embodiment, patient care devices are associated with a domain, and some or all of the open architecture-related functions of the devices are limited by domain. In some embodiments, devices perform context management, maintaining information related to one or more patients or other information related to the context of the devices. In some embodiments, the domain is related to or based upon the context information. In an embodiment, patient care devices and other devices are configured to determine whether a minimum set of devices is present on the network or domain.

One aspect of the invention includes one or more management servers connected to patient care devices via an open architecture system. The server may provide functions including data storage, logging, transactions, rules management and execution, redundancy, failure monitoring, communication with end-user devices, and other functions, as well as any combination of these functions or all of these functions.

One aspect of the invention includes a management system, which may be a management server or a separate device. The management system may provide functions including a configuration database and remote access.

One aspect of the invention includes automatic preference management based on proximity sensors, in which a device detects the presence of a person or other entity in the proximity of the device and adjusts preference settings on the device itself and/or other devices based on stored preference data.

Other aspects and embodiments of the invention are disclosed throughout this specification and in the drawings.

### BRIEF DESCRIPTION OF THE DRAWINGS

FIGS. 1A and 1B illustrate embodiments of a patient care system using a virtual machine.

FIGS. 1C and 2 illustrate examples of patient care networks.

FIG. 3 illustrates a typical pulse oximeter with display screen.

FIGS. 4-6 illustrate pulse oximeters according to several embodiments displaying messages.

### DETAILED DESCRIPTION

Various embodiments according to the invention will be described hereinafter with reference to the accompanying drawings. These embodiments are illustrated and described by example only, and are not intended to limit the scope of the invention.

A patient care system of certain embodiments includes one or more patient monitoring devices or other patient care equipment connected to a shared network using open architecture communications standards. The patient monitoring devices or other patient care equipment of certain embodiments includes a physiological monitor or other patient care equipment coupled with a network interface module. The physiological monitor can include one or more sensors and a sensor processing module for processing signals from the sensors. The network interface module receives physiological information or other communications from the patient care equipment and transmits this information over the shared network. The network interface module also receives communications from other equipment on the system and delivers the communications to the patient care equipment. The network interface module may connect to a variety of physiological monitors or patient care equipment.

In certain embodiments, the network interface module facilitates establishing a network connection directly with end users over the shared network. These end users, including doctors, nurses, and other hospital staff, may receive physiological information, alarms, and alerts from the network interface module on an electronic device, such as a pager, PDA, laptop, computer, computer on wheels (COW), or the like. In addition, in an embodiment, the end users can also send instructions and other communications to the patient care equipment.

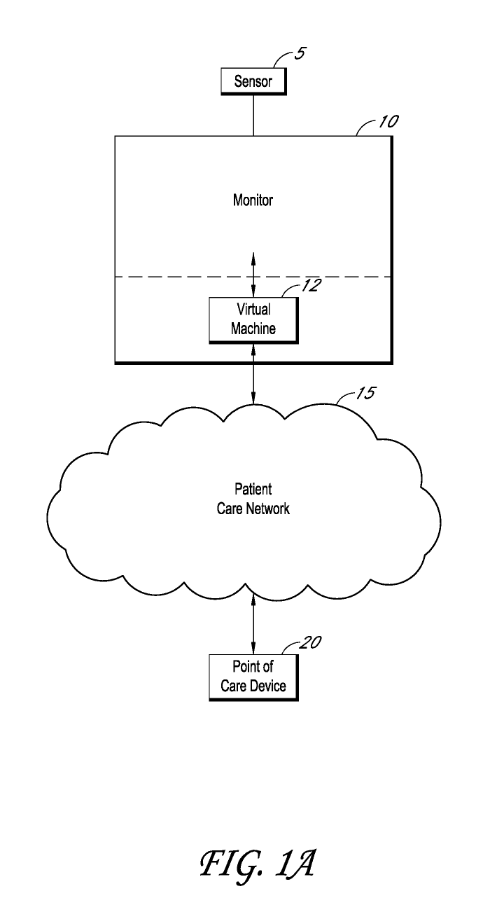

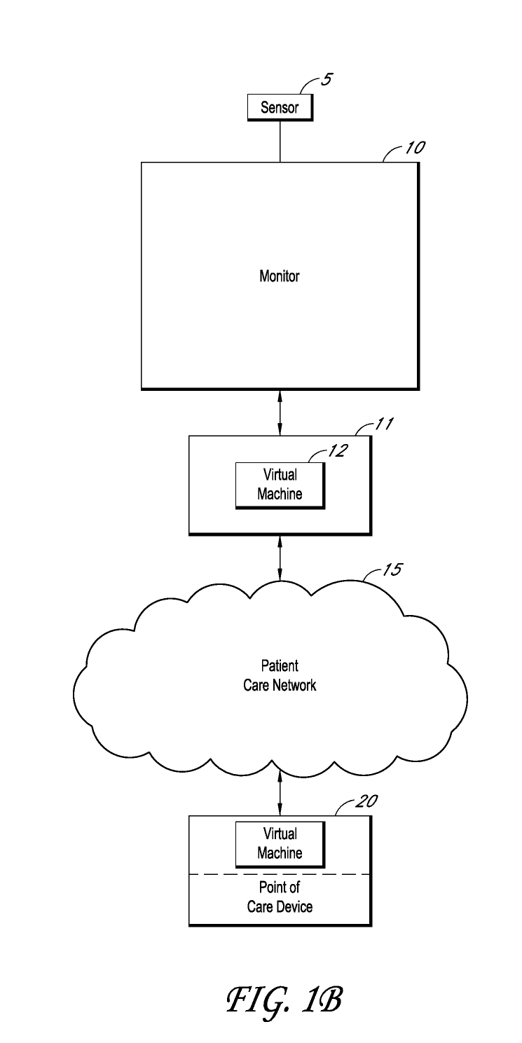

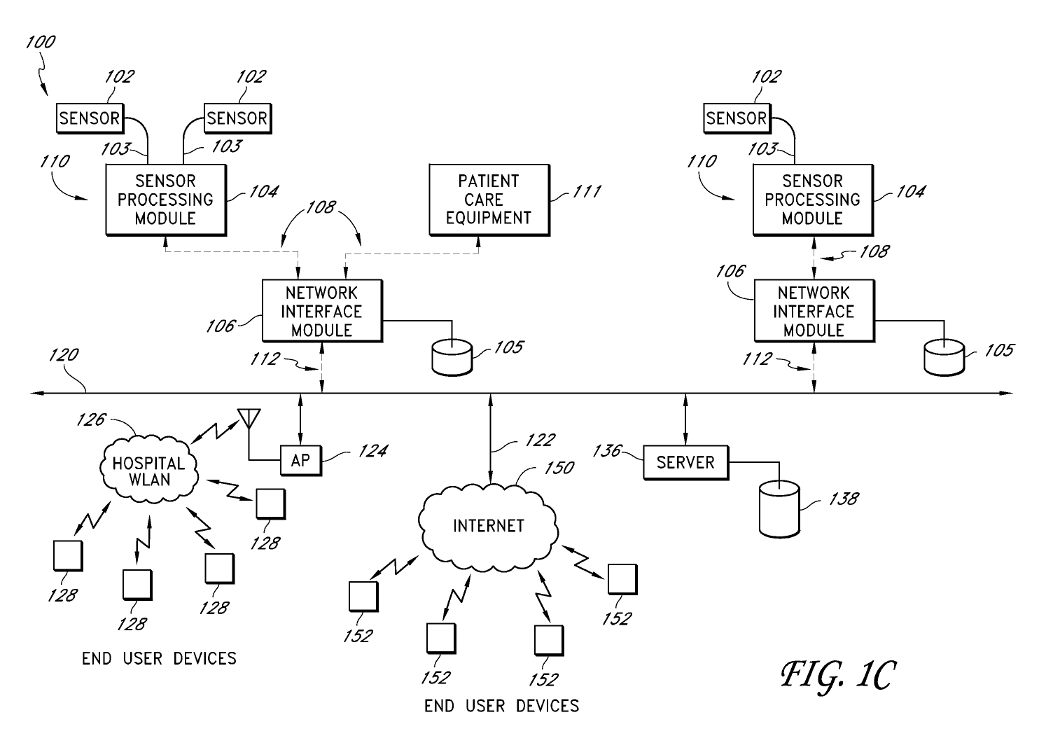

FIG. 1A illustrates a patient monitor 10 connected to a sensor 5 for receiving signals indicative of a physiological condition of a patient. The monitor can include a processor running software configured to process and/or analyze the signal to determine the physiological condition of the patient. The monitor can be running an operating system or other software configured to manage the processing of the signal on the monitor 5 processor. In one embodiment, the sensor 5 comprises an electromagnetic wave emitter that emits waves of one or more particular wavelengths, optionally additional emitters for different wavelengths, and one or more optical sensors that detect electromagnetic waves emitted from the emitter or emitters, where the emitted electromagnetic waves are reflected by, transflected through, and/or transmitted through tissue of a patient, such as the skin of the patient. The sensor 5 may be configured to transmit raw measurement data to the processor. The sensor 5 and/or the processor may be further configured to perform transformations, analyses, and/or calculations on the transmitted data. This type of sensor is known in the art of pulse oximetry devices and other patient monitor devices, and its implementation is well known to those of ordinary skill in those arts. The sensor may also be an ECG sensor, acoustic sensor, hemoglobin sensor, or other type of sensor. It is contemplated that, in some embodiments, different types of sensors and/or multiple sensors of the same type will be usable within a system.

In an embodiment, patient monitors such as those shown in FIG. 1A can include a virtual machine, such as virtual machine 12. The virtual machine 12 can include hardware and/or software, for example it could include one or more software modules running on the monitor's 5 hardware. In an embodiment, the virtual machine and monitor software operate in conjunction with a hypervisor. In an embodiment, the virtual machine runs on the monitor's 5 operating system or a combination of an operating system and hypervisor. Alternatively, the functions described in the present application with respect to a virtual machine can operate on the systems main software platform. For example, in some embodiments, the virtual machine can be an application running on the patient monitor or other patient care device that runs in conjunction with other software running on the patient care device.

In an embodiment, the virtual machine is configured to abstract out and translate measurements, instructions, alarms, management services and other communications into an open architecture specification which is compatible with the rest of the care facility's network 15 and/or a point of care device 20. The point of care device is, for purposes of this disclosure, any device accessed by a care giver. This allows multiple patient monitoring systems operating on different software platforms with different communications protocols to operate with each other in a universal, efficient and coherent manner. For example, in an embodiment, various aspects of the sensor(s) 5, such as, for example, a sensor off alert, an expiration alert, an on/off signal, or the like, is abstracted into a standard format for communication. Various other aspects of the devices can also be abstracted as described in greater detail below. The virtual machine can also be configured to translate instructions received from the care facility network. The virtual machine can translate instructions received into a form which is understood by the patient monitor or patient care device.

Virtual machines can be adapted to each individual device. Alternatively, one or more virtual machines can be running on a separate device or network location in communication with each patient monitor or patient care device. In an embodiment, both the patient care devices, including patient monitors, and the point of care devices used by the care facility staff operate using virtual machines. In other words, a virtual machine can be running on every device on the network, with the possible exception of devices which merely pass the information along such as routers, switches, hubs, etc.

FIG. 1B illustrates a sensor 5 and monitor 10 which communicate with a separate device 11 including the virtual machine 12. Monitor 10 and device 11 communicate through any type of communication methods included on the monitor 10, such as, for example, serial communications, wired or wireless Ethernet, or the like. The virtual machine then manages and translates communications between the monitor 10 and the rest of the care facility's network 15.

In an embodiment, the virtual machine abstracts out device specific information and formats the information into a form usable and understandable by other devices on the patient care network. For example, the virtual machine can abstract out the following categories of information: core monitoring services, special services, low level services and content management services. Although described in relation to certain categories, a person of skill in the art will understand from the disclosure herein that other or different categories or category names can be used and the present description is meant by way of example and not limitation.

The core monitoring services include sensor management, measurement engine management, device management, connectivity management and alarm engine. The sensor management includes services related to sensor channels, channel errors (such as, for example, sensor off, sensor expired, calibration information or the like) and channel exceptions. The measurement engine management includes services related to measurement raw type, measurement limits, alarm levels, alarm priority, numerics including waveforms and measurement numbers, multiple channel i/o, sampling interval, display attributes, multiple levels of alarm thresholds, averaging, 3D alarms, or the like. The device management services include services related to system faults, configurations and settings, accounting, performance, and security (such as, for example, authentication, integrity, privacy and the like). The connectivity management provides services related to interface connections and the alarm engine manages activation and status of alarms.

The special services include location and presence sensing, connectivity management for other local device, such as, for example, bedside integration, integration with body worn devices or sensors, cameras, speakers, video displays, etc. Special services also include power and hosting services and display access services.

The low level services include time services, which, for example, provide a fine grain time and clock sync service. Other low level services include name services and spaces including directories, rules, roles, privileges and scope. The low level services also include a log service. Low level services can include services which are partitioned with higher security levels and limited access rights to prevent tampering or accidental disruptions. For example, the partitions can include protected measurement namespaces that prevent or attempt to prevent one monitoring module from influencing another (e.g., even when both modules use the same naming conventions). Namespaces may be predefined and/or may be generated dynamically. In response to a new measurement module being provided, for instance, a new namespace may be generated for that measurement module. Namespaces can be automatically generated even when modules from different manufacturers are provided.

Content management services include services related to pushing and pulling externally originating content. This can include, such as, for example, messages (text, voice, data, video) both in and out. This allows, for example, advertisements to be displayed or played on a monitoring device. This also allows for direct communications between a care giver at a care center and a patient at a remote (e.g. home) location. For example, a care giver can recommend a treatment to a patient which is displayed on the patient monitor in the patient's home.

In an embodiment, a high precision time sync is provided between the measurement modules and the point of care. The high precision time sync may be provided by hardware and/or software timing modules. Each measurement module can have one or more physiological measurement channels receiving signals from one or more physiological sensors. This allows clocks across multiple measurement modules to be synchronized allowing for synchronization in time sensitive physiological measurements.

In an embodiment, one or more levels of synchronization are provided. In one embodiment, time synchronization between the measurement modules and the point of care instrument is provided. This synchronization can provide forward time sync of about 100 micro seconds or less resolution. In addition or alternatively, time synchronization between the main processor and the ADC clock on the measurement module is also performed. In an embodiment, the synchronization between the measurement module and the point of care is used to synchronize the main processor clock and the ADC of the measurement module.

In an embodiment, time synchronization is performed using an external or reference clock. This can be done using a time server, a standards organization based time source (e.g. NIST, NOA, etc.), GPS clocks, a radio broadcast clock or the like. In an embodiment, the reference clock is used to synchronize one or both of the measurement module and the point of care module. In an embodiment, time sync information is provided via a communication port or a clock sync trigger line when available.

In an embodiment, each measurement module can be uniquely identified by a factory assigned identifier. The available physiological measurements from the measurement module are determined by a number of factors including (i) what the measurement module is capable of measuring; (ii) what it has been configured to measure; (iii) what are the connected sensors and probes that are actually installed (iv) the measurements that are being actively measured at any given point in time. These identity elements need to be established only after a measurement module has been authenticated as a valid module with the point of care instrument.

In one embodiment, the sensing functions of a patient monitoring device 110 are decoupled from post-processing functions of the device, so that post-processing functions can be relocated to other devices. In an embodiment, this relocation feature can be implemented in conjunction with the virtual machine features described elsewhere in this specification, and the relocation feature can be implemented using the virtual machine features.

Sensing functions can include receiving data from sensors 102 and/or basic computations or transformations using that received data. Post-processing functions can include computing waveforms using sensor data, calculating averages or other statistics, displaying information about the data on a visual or other display, producing alerts or warnings on the device, or broadcasting data to data monitors such as pagers. Other sensing and post-processing functions are described throughout this specification.

Ordinarily, a patient monitoring device will perform both the sensing functions and the post-processing functions. However, it can be advantageous at times to have the sensor perform the sensing functions only, while a second device performs the post-processing functions. This can be useful, for example, in situations where a battery-operated device may be monitoring a patient parameter levels and displaying a real-time graph of the measured parameter levels. If the battery of the device begins to run low, it can be advantageous to shut off the monitor's display but continue to operate the parameter acquisition only in order to continue measuring the parameter while conserving battery. This allows the device to extend its battery life while still monitoring the patient. In an embodiment, the monitor will continue to provide alerts and can turn its display on when an alert is sounded. In another embodiment, the post processing functions, such as the display, can be distributed to another device on the network. In alternative situations, another device can be connected to the network, for example, with a larger display or central monitoring functions, and it can be advantageous to transfer the graph display to this other device.

In order to provide this post processing offloading, the question of whether to relocate the post-processing function must first be determined. In some embodiments, the patient monitoring device itself detects a need to relocate a post-processing function. For example, the device can detect that its battery is low, and send out an alert over a network or by other means to other devices. In other embodiments, an external device, such as a monitoring server, detects the need to relocate the post-processing function. Thus, the need to relocate the post-processing function can result from the patient monitoring device itself (e.g., the device experiences a fault or failure), it can result from another device (e.g. a device with better resources becomes available), or it can be dictated by an external user via a command.

Second, a rule is identified that determines the action to be taken in response to the need to relocate the post-processing function. The rule can be stored on the patient monitoring device, on a monitoring server, or on any other computer storage medium. For example, in one embodiment, the rules are stored and governed by a master device in a master/slave architecture system. Typically the rule will incorporate executable instructions, or the rule will be read by a computer program and direct the execution of the program. The rule can take, as input, information about the patient monitoring device and/or other information available on the network. The rule(s) can determine, among other things, whether to relocate the post-processing function, where to relocate it to, and how the relocation is to be done.

In some embodiments, relocation of the post-processing function from the patient monitoring device to a second device is performed according to the following method or variants thereof. The patient monitoring device disables the post-processing function on itself. The patient monitoring device continues to operate the sensing functions, and it transmits data based on the sensing function across a network. The data can be transmitted directly to the second device, or it can be transmitted to a central server or any other device, which will then forward the data to the second device. The second device enables the post-processing function, receives the data, and performs the post-processing function using the received data.

Relocation of post-processing functions is not limited to handling device faults or failures. In some embodiments, for example, post-processing functions can be reallocated to devices with greater processing power. Additionally, post-processing functions can be reallocated to multiple devices, or the post-processing functions can be performed by the original patient monitoring device in conjunction with or in addition to one or more other devices. Thus, in an embodiment, the post-processing functions are redundantly performed, creating greater stability and reliability for the system. In some embodiments, the device monitoring functions can similarly be made redundant across multiple devices.

Decoupling and sharing post-processing functions thus provides for automatic or manual sharing of resources as needed or required among the devices on the network. This provides a redundancy to the collection of patient monitoring devices incase of failure, effectively creating a single patient monitor out of many individual monitors. This can occur, for example, between devices that are not normally considered to provide such functionality. Central monitors, for example, are known in the art for receiving and displaying parameters obtained from individual patient monitors. However, the present disclosure allows, for example, an SpO₂ monitor to offload its display functions onto a ventilator monitor's screen or the screen of any other patient monitor in the network. This can be done, for example, by cycling the ventilator's monitor between the ventilator's originating display and the SpO₂'s originating display. In an alternative embodiment, the ventilator's display can be reconfigured to display both ventilator parameters and SpO₂ originating display. Alternatively, or in addition to offloading display features, as described above, other post processing functions can also be offloaded.

In an embodiment alarms can be shared to provide a greater probability that they will be notice and acted upon by a care giver. For example, if an SpO₂ alarm is triggered, the alarm can also be displayed or audibly provided by the ventilator device. In an embodiment, a triggered alarm can be shared among multiple devices. For example, an SpO₂ alarm can be visually or audibly provided by a ventilator device, a pulse monitor, and any number of other devices, based on rules stored in any of the devices or one or more servers. This provides for redundancy of alarms.

In an embodiment, some or all non-essential functionality can be pushed to a single device or group of devices anytime the other devices are available in order to free up resources on one or more monitors and conserve battery.

FIG. 1C illustrates another embodiment of a physiological monitoring system 100 including an open architecture system as described above. This architecture in various implementations is a shared, or open, network which includes multiple patient monitoring devices 110, a network bus 120 (e.g., an Ethernet backbone), and a hospital WLAN 126. In addition, the shared network may further include a connection 122 to the Internet 150 or other networks, to end user devices 152 over the Internet 150, and to end user devices 152 over the hospital WLAN 126. The physiological monitoring system 100 of certain embodiments' is therefore an enterprise system that achieves a cost-effective replacement for currently available patient monitoring systems.

The physiological monitoring system 100 includes a plurality of bedside devices, e.g., patient monitoring devices 110 and/or patient care equipment 111. The patient monitoring devices 110 of various embodiments include sensors 102, one or more sensor processing modules 104, and a communications module, e.g., network interface module 106. In an embodiment, the network interface module can be built into or form part of the patient monitoring device 110 or patient care equipment 111. In an embodiment, the network interface module is a separate or stand alone piece of hardware which can be configured to communicate with one or more patient monitoring devices 110 or patient care equipment 111. In the depicted embodiment, a patient monitoring devices 110 and patient care equipment 111 are shown. One patient monitoring device includes sensor 102, sensor processing module 104, and network interface module 106. The other patient monitoring device 110 includes two (or more) sensors 102. A person of skill in the art will understand from the present disclosure that any number or combination of sensors, sensor processing modules, or patent care equipment can be used with the presently disclosed system.

In certain embodiments, each patient monitoring device 110 or other patient care equipment are used by one medical patient. The patient monitoring devices 110 and patient care equipment 111 form a network of patient care devices, each of which can communicate with clinicians and other end users over a shared network, including a hospital network 126 and network interfaces to the Internet 150. The network may use standard communications protocols, such as Ethernet, TCP/IP, 802.11b/g/n, IPX/SPX, Appletalk, PPP, and other protocols known to those skilled in the art. As will be understood by a person of skill in the art from the present disclosure, a single piece of patient care equipment or patient monitoring device can also be used by multiple patients or switched between patients.

One or more sensors 102 of the patient monitoring device 110 are attached to a medical patient. These sensors 102 may include ECG sensors, acoustic sensors, pulse oximeters, and other types of sensors. The sensors 102 obtain physiological information from a medical patient and transmit this information to the sensor processing module 104 through cables 103 or through a wireless connection (not shown). In certain embodiments, the physiological information includes one or more physiological parameters or values and waveforms corresponding to the physiological parameters.

The sensor processing module 104 receives physiological information from the sensors 102. The sensor processing module 104 of certain embodiments includes a circuit having a processor, input ports for receiving the physiological information, software for processing the physiological information in the processor, an optional display, and optionally an input device (e.g., a keyboard). In addition, the sensor processing module 104 contains one or more output ports, such as serial ports. For example, an RS232, RS423, or autobaud RS232 (serial interface standard) port or a universal serial bus (USB) port may be included in the sensor processing module 104. Patient care equipment 111 can likewise include input and output interfaces for receiving and transmitting communications and instructions.

In certain embodiments, the sensor processing module 104 generates waveforms from signals received from the sensors 102. The sensor processing module 104 may also analyze single or multiparameter trends to provide early warning alerts to clinicians prior to an alarm event. In addition, the sensor processing module 104 in certain embodiments generates alarms, otherwise known as faults, failures, or alerts, in response to physiological parameters exceeding certain safe thresholds.

Example alerts include no communication with pulse oximeter, alarm silenced on pulse oximeter, instrument low battery (pulse oximeter), and transmitter low battery. Example alarms include SpO₂ levels and alarms, high and low SpO₂, high and low PR, HbCO level and alarms, HbMET level and alarms, pulse rate and alarms, no sensor, sensor off patient, sensor error, low perfusion index, low signal quality, HbCO, HbMET, PI trend alarm, and desat index alarm.

The network interface module 106 in the depicted embodiment is connected to one or more sensor processing modules 104 or patient care equipment 111 through one or more connectors 108, which may be serial connectors corresponding to the serial ports in the sensor processing modules 104. Alternatively, the connectors 108 may be any hard wired or wireless communications types including wired or wireless Ethernet, telephone lines, Wi-Fi, etc. Dashed lines on the connector 108 indicate that the network interface module 106 of certain embodiments is not permanently attached to the sensor processing modules 104. In alternative embodiments (not shown), however, the network interface module 106 is contained within a sensor processing module 104 or patient care equipment 111.

The network interface module 106 in various implementations includes a processor, an input port (such as a standard RS232 serial port, Ethernet port, wireless transceiver, etc.), a network output port such as an Ethernet port, Ethernet transceiver serial interface, etc., and software which enables the network interface module 106 to act as a network-communications enabled device. In addition, the network interface module 106 includes a storage device 114, which may be included within the network interface module 106 or attached separately to the network interface module 106.

The network interface module 106 manages the connectivity overhead for initiating and maintaining connectivity with end user devices over the shared network. In certain embodiments, the network interface module 106 manages connectivity by acting as a microserver or web server. In such instances, the network interface module 106 is a network connection enabled device. As a web server, the network interface module 106 establishes direct connections to the Internet 150, such that an end user may access web pages stored on the storage device 105 of the network interface module 106. In an embodiment, the network interface module 106 therefore does not require a separate server for connecting to the Internet 150. In one embodiment, the network interface module 106 connects to the Internet 150 directly through a modem, such that the connection 122 includes a modem. In managing connectivity over the shared network, the network interface module 106 may also perform security management functions, such as user authentication. In an embodiment, as described in more detail below, the network interface module 106 can include a patient care equipment virtual machine configured to receive and communicate instructions and other communications with one or more patient monitors 110 and one or more patient care equipment 111.

In certain embodiments, the network interface module 106 sends data over the shared network through an access point 124 or other wireless or wired transmitter. Alternatively, the network interface module 106 may communicate directly with end users over the Internet 150. End users such as clinicians carrying notifier devices, e.g., end user devices 128, 152 connected to the hospital WLAN 126 may receive real-time viewing of physiological patient parameters and waveforms on demand or in the event of an alarm or alert. End users can also provide instructions or other communications to the patient monitor 110 or patient care equipment 111 using the same end user devices. Real-time or slightly delayed transmission of physiological information in certain embodiments comports with standards for alarm latency in compliance with Joint Commission on Accreditation of Healthcare Organizations (JCAHO) standards for effective alarm response. The network interface module 106 of certain embodiments therefore adds functionality equivalent to a central nurses' station.

In certain embodiments, the network interface module 106 performs context management. In one embodiment, context management includes associating context information with physiological information to form a contextual data package. Context information may include several categories of information, including the categories of context information related to the network interface module 106, context information related to the medical patient, context information related to usage of the network interface module 106, and context information related to a network connection. Within one or more of these context categories, context information might include a patient name, a patients' unique hospital identification number, patient location, an identification number for a network interface module 106, time stamps for events occurring in the physiological monitoring system 100, environmental conditions such as changes to the state of the network and usage statistics of the network interface module 106, and identification information corresponding to the network link (e.g., whether the network connection is WiFi or Ethernet). In one embodiment, the context information in the contextual data package may include all of or any subset of context information from one or more of the context categories. In an embodiment, the context information and measurement information are packaged by the virtual machine.

The network interface module 106 receives context information, for example, by a nurse entering the information in the network interface module 106 or from a server 136. In one embodiment, by receiving this information (including, e.g., patient identification number and location), the network interface module 106 becomes exclusively assigned to the medical patient. The network interface module 106 transmits or communicates the contextual data package to clinicians during an alarm or alert, upon clinician request, or on a scheduled basis. In addition, the network interface module 106 may transmit a continuous stream of physiological information to clinicians.

By optionally connecting to multiple sensor processing modules 104 in certain embodiments, the network interface module 106 is able to associate patient context information and other context information with multiple sensor processing modules 104. Consequently, context can be created for one or more sensor processing modules 104 in addition to context being created for the network interface module 106.

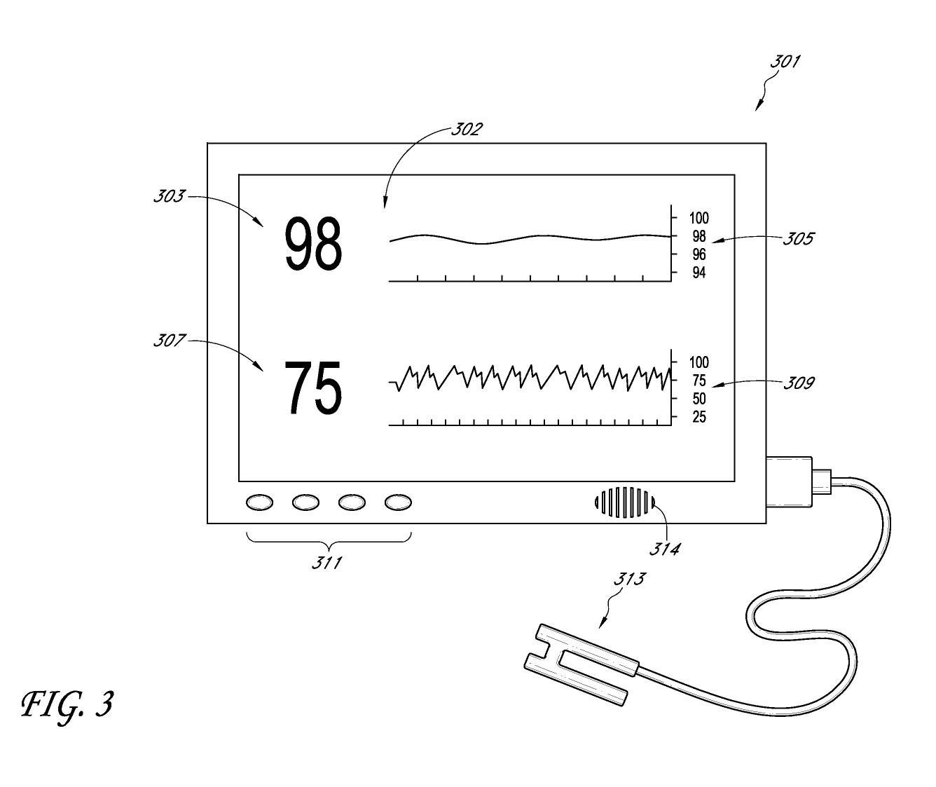

In addition to transmitting the contextual data package, the network interface module 106 in one embodiment stores the contextual data package in the storage device 114. The storage device 114 may be a flash memory, a hard disk drive, or other form of non-volatile or volatile memory. In certain embodiments the storage device 114 acts as a flow control buffer. The network interface module 106 uses the storage device 114 acting as a flow control buffer to perform flow control during communications, as explained more fully below in connection with FIG. 3.

In some implementations, a server 136 may optionally be included in the physiological monitoring system 100. The server 136 in these implementations is generally a computing device such as a blade server or the like. In certain embodiments, the server 136 is an appliance server housed in a data closet. In other embodiments, the server 136 is a server located at a central nurses' station, such as a workstation server. In still other embodiments, the server may be a patient monitoring device.

The server 136 receives contextual data packages from a plurality of network interface modules 106 and stores the contextual data package in a storage device 138. In certain embodiments, this storage device 138 therefore archives long-term patient data. This patient data may be maintained even after the patient is discharged. In storing patient data, the server 136 may act as an interface between the shared network and an external electronic medical record (EMR) system.

The server 136 may also store data concerning user interactions with the system and system performance metrics. Integrated into the server 136 of certain embodiments is a journal database that stores every alert and alarm or a subset of the alerts and alarms as well as human interaction in much the same way as an aviation “black box” records cockpit activity. In an embodiment, the journal is not accessible to the clinical end user and, without technical authorization, cannot be tampered with. In addition, the server 136 may perform internal journaling of system performance metrics such as overall system uptime.

In one embodiment, the journaling function of the server 136 constitutes a transaction-based architecture. Certain transactions of the physiological monitoring system 100 are journaled such that a timeline of recorded events may later be re-constructed to evaluate the quality of healthcare given. These transactions include state changes relating to physiological information from the patient monitoring devices 100, to the patient monitoring devices 110, to the hospital WLAN 126 connection, to user operation, and to system behavior. Journaling related to the physiological information received from a physiological monitor in one embodiment includes recording the physiological information itself, recording changes in the physiological information, or both.

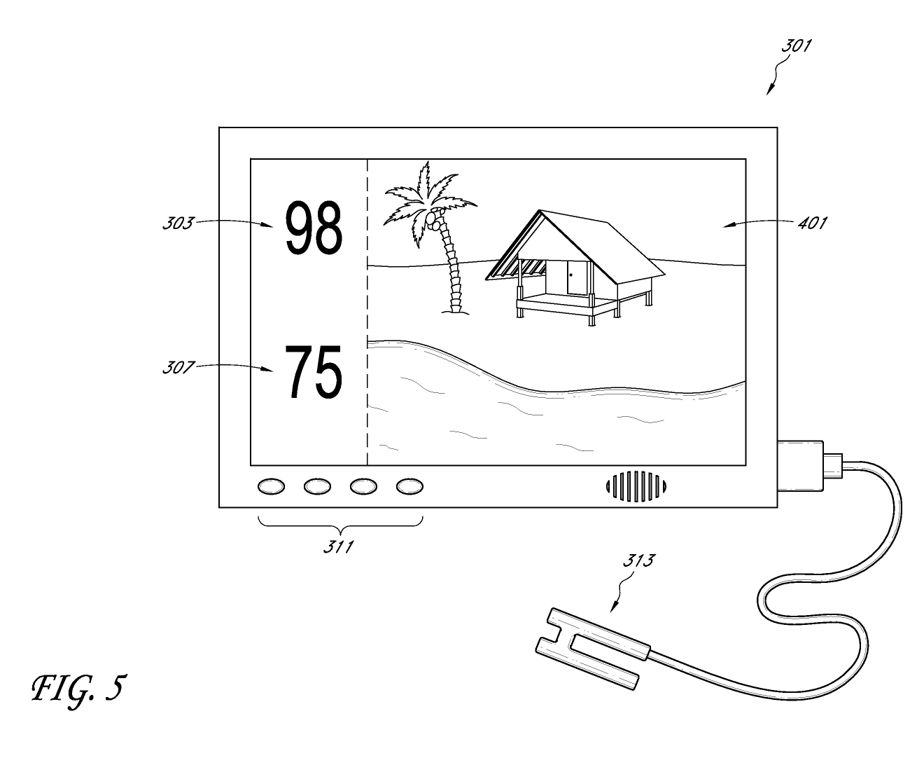

The server 136 in certain embodiments provides logic and management tools to maintain connectivity between network interface modules 106, clinician notification devices such as PDAs and pagers, and external systems such as EMRs. The server 136 of certain embodiments also provides a web based interface to allow installation (provisioning) of software rated to the physiological monitoring system 100, adding new devices to the system, assigning notifiers (e.g., PDAs, pagers, and the like) to individual clinicians for alarm notification at beginning and end of shift, escalation algorithms in cases where a primary caregiver does not respond to an alarm, interfaces to provide management reporting on the alarm occurrence and response time, location management, and internal journaling of system performance metrics such as overall system uptime (see, e.g., FIG. 5 and accompanying description).

The server 136 in certain embodiments also provides a platform for advanced rules engines and signal processing algorithms that provide early alerts in anticipation of a clinical alarm. The operating system on the server 136 in one embodiment is Linux-based, though a Microsoft-based, OSX-based or other operating system may also be used. Moreover, the server 136 is expandable to include data storage devices and system redundancy capabilities such as RAID (random array of independent disks) and High Availability options. The server 136 can also operate the virtual machine to facilitate proper communications between network devices, patient care devices and point of care devices.

In one embodiment, the open architecture of the network enables the server to communicate with devices of different types, while differentiating between the types of devices present on the network. The devices, possibly through use of a virtual machine, are able to communicate information describing their nature and features, to other devices on the network. The rules engines present in the server or possibly other devices can utilize this descriptive information to provide specialized responses to alerts, alarms, faults, or other events of interest. These rule-based specialized responses may be specific to particular devices or particular types of devices, and the responsive actions taken may depend on other devices connected to the network and/or associated with a particular domain or context. The rules may designate, as responsive actions, any of the various actions described throughout this specification, and additional possible responses will be known to those of ordinary skill in the art.

One embodiment includes multiple servers performing the same or similar functions described above. Each server may separately operate rules engines to anticipate or detect alarm conditions, provide logging and context management services, and so on. Multiple servers introduce redundancy into the system, making it more tolerant of failures in a single server. For example, if one server loses communication with the network, other servers may be able to continue performing operations. In some embodiments, each server monitors medical devices connected to the network. In some embodiments, each server additionally monitors the other servers and signals an alert or other warning if one of the other servers becomes inoperable or otherwise experiences a failure. This way, the servers create a self-monitoring system that makes failures highly unlikely to go undetected.

When a server detects a failure in a medical device, another server, or itself, the server may perform a number of functions to handle the problem. The server may produce an audible or visual alarm to alert hospital staff of the problem. The server may send out an alert via a network communication, to a pager, email account, or other notifier devices or recipients, some examples of which are given throughout this specification. The server may also distribute instructions to other connected devices to compensate for the error, such as by the post-processing function relocation method described elsewhere in this specification.

In another embodiment (not shown), end user devices 128, 152 include one way POCSAG Pagers having a 2 line display with audible and vibrate mode, of suitable size and durability for severe mechanical environments typical of hospital general floor settings. In yet another embodiment, the end user devices 128, 152 include two way paging systems, such as Motorola Flex and WLAN pagers. One advantage of two-way paging is the ability to confirm message receipt and the ability to remotely silence alarms. Wireless PDAs may also be used by end users based on ruggedness and acceptable form factors as determined by an end user. An example of such a device is the Symbol Technology MC50 PDA/Barcode Scanner.

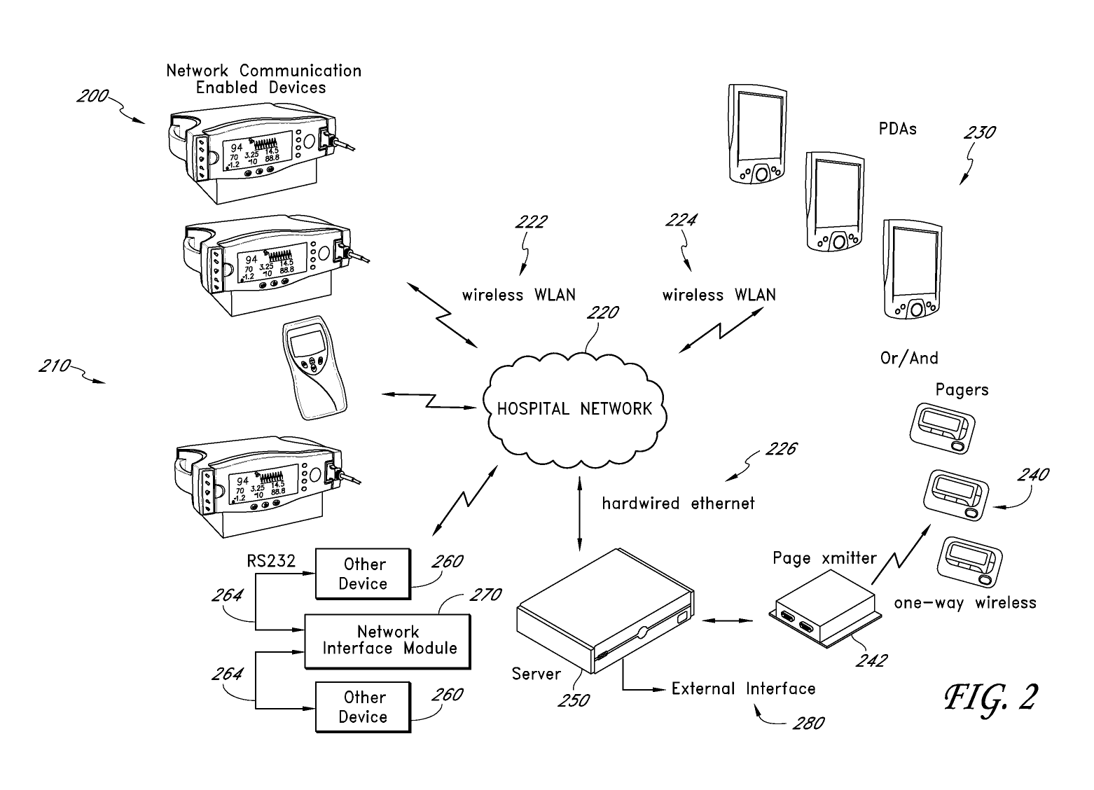

FIG. 2 depicts another embodiment of a physiological monitoring system 200 of the present invention. The physiological monitoring system 200 (or alternatively other patient care equipment, not shown in FIG. 2) includes network communications enabled devices 210. The network communications enabled devices 210 are connected directly to a hospital network 220 through a wireless connection. In certain embodiments, the network communications enabled devices 210 include sensors and sensor processing modules, similar to the sensors 102 and sensor processing modules 104 of FIG. 1. Certain of these network communications enabled devices 210 are bedside devices, and others are handheld or otherwise patient-worn devices that may be used by an ambulatory (mobile) patient.

The hospital network 220 transmits physiological information and context information to clinician notifier devices, including pagers 240, PDAs 230 as well as cell phones, portable and stationary computers or any other communications devices. In certain embodiments, the hospital network 220 utilizes a server 250 to transmit contextual data packages to a page transmitter 242, which further transmits the data to one-way wireless pagers 240. An external interface 280 may be coupled with the server 250. The external interface 280 could include one or more of the following: enterprise paging, nurse call systems, wide area paging systems, enterprise clinical and patient information systems, and third party monitoring and surveillance systems.

Certain other devices 260, such as some patient monitoring equipment, are not network communications enabled devices. That is, these other devices 260 are unable to connect to a network unaided. In the depicted physiological monitoring system 200, example devices 260 that are not network communications enabled are connected to a network interface module 270. The network interface module 270 is connected to the non-network communication enabled other devices 260 through RS232 cables 264. Such a connection is a standardized serial connection found on many devices. Because the network interface module 270 has an RS232 port, the network interface module 270 can allow non-network communication enabled patient monitoring devices to connect directly to the hospital network 220 and also to the Internet.

Moreover, by connecting to one or more other devices 260 in some embodiments, the network interface module 270 is able to associate patient context information and other context information with one or more other devices 260. Consequently, context can be created for one or more other devices 260 in addition to context being created for the network interface module 270.

In certain embodiments, content between the network components is managed. As described above, in an embodiment, various types of messages, advertisements or other content can be communicated and displayed or played on the patient care equipment or point of care devices. This content can originate with the patient care devices and point of care devices or it can originate outside of these devices. The content is managed so as to provide and preserve security concerns including authentication and integrity.

In addition, content communications must also be managed to preserve measurement integrity and priority, patient privacy, and other concerns unique to patient care facilities. In an embodiment, firewalls, security token authentication systems, pass keys, combinations of the same, and the like are used to protect the integrity of the system. In an embodiment, addresses and memory space is partitioned and dedicated to core functionality in order to block intended or accidental interference with the core functionality. Thus, a user may be able to change certain functionality at a higher level but will be restricted from changing core functionality and disrupting the critical operations of the system.

In some embodiments, the hospital network 220 is divided into domains. Each domain may be associated with a single patient, a clinician, a hospital ward, or a department and other arrangements for domains are possible. Alternatively, the entire hospital may serve as a single domain. In some embodiments, domains are based on context information described elsewhere in this specification.

Each domain may have one or more servers 250, or a server may be shared among several domains. Devices on the hospital network, such as 210, 260, and 270, are typically associated with a single domain, although a device may be assigned to more than one domain in some embodiments. These assignments to domains may be made when the device starts up or during the operation of the server. The assignments may be made manually, by a hospital staff member entering the domain information directly onto the devices for example, or the assignments may be made automatically, through a network self-discovery protocol such as DHCP or SMB, for example. In some embodiments, device domains may be determined, in whole or in part, based on the location of the device. This may be done using, for example, a network of proximity sensors. Using location to determine the domain of a device means that devices can easily be moved between domains simply by physically relocating the devices. Alternatively proximity to a patient or assignment of a device to a patient serves to designate the domain. This can be done through manual entering of information on a monitor or it can be done through the use of RFID tags of a patient or other methods of automatically determining that the devices are used for a particular patient.

In an embodiment, some or all of the functions of devices are limited to the domain associated with the device. For example, the post-processing function relocation, fault monitoring, data communication, transaction logging, and other features described throughout this specification, or any subset of those functions, may be limited by domains. This may be used, for example, to ensure that devices monitoring one patient do not affect devices associated with a second patient, to prevent confusion in recordkeeping and management of the individual patients.

In an embodiment, devices may perform an initialization routine upon starting up or upon first connecting to a hospital network, including the following steps. The device detects the network that it will connect to, and it connects to the network. Once the device is connected, it optionally transmits data across the network pertaining to the device. This data may be transmitted upon a request received from another device, such as a central management server, or it may be broadcast by the device upon its own initiative. The data transmitted might include information describing the device's type and capabilities, a unique identifier for the device, the domain with which the device is associated, and/or other devices that this device requires for operation. Because of the open protocols used on the hospital network, other compatible devices will be able to receive this data and register the device as being available on the network.

In some cases, a device may require other devices to be present and operating on the same network or domain. For example, a display monitor that shows a graph of a patient's heartbeat and blood pressure cannot operate unless there is also a pulse monitor and a blood pressure monitor present on the network and in the same domain. To solve this problem, devices in some embodiments of the invention maintain one or more rules defining a minimum set of related devices needed for operation. Upon starting up, the device receives information about other devices present on the network. The information may be pushed to the device automatically from other devices or from a central server, or it may be received in response to a request for information sent by the device. Once the information about other devices is received, the device compares the information with its rule defining the minimum set of related devices. If the rule is satisfied, then the device proceeds with operation. If the rule is not satisfied, then the device may produce an alert or warning, or it may transmit information through the network, to inform hospital staff of the situation.

In some embodiments, the required device analysis may be performed by another device, such as a central server. In some embodiments, the required device analysis is performed continuously, at specific intervals, or upon specific events such as the addition or removal of a device to the network or domain, in order to ensure that the minimum set of devices is consistently present. Thus, the rules may not only be used to determine which devices must be present at startup, but also which devices can be removed or moved around within the network.

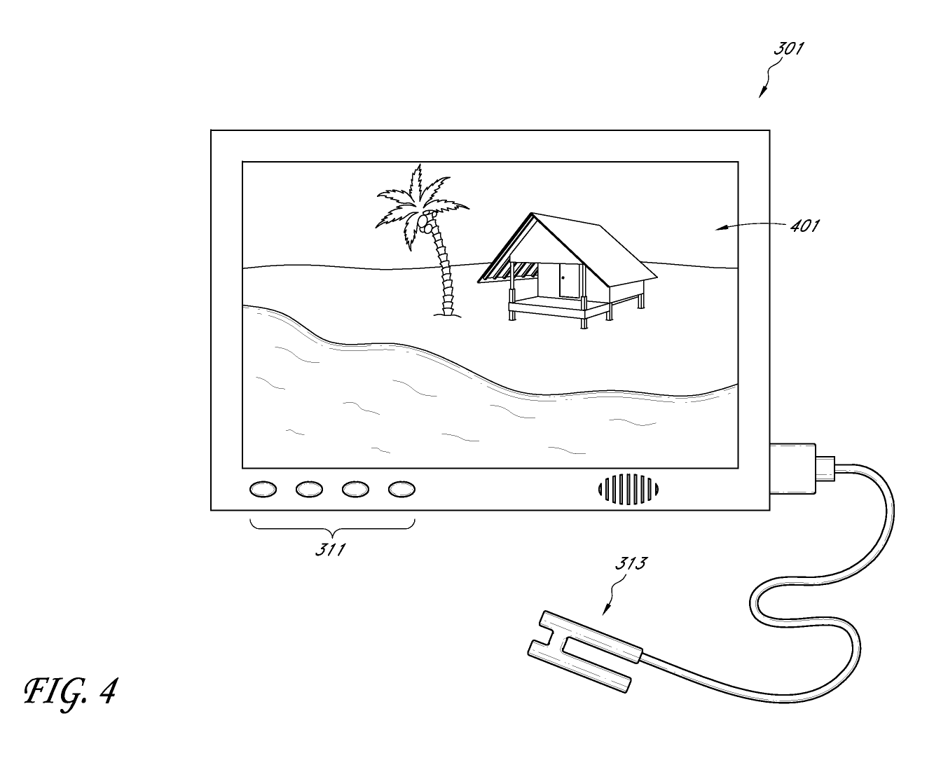

In an embodiment, a message, such as, for example, a message from a physician to a patient or vice versa or an advertisement can be displayed on a patient care device. In an embodiment, a camera or webcam setup can be used to communicate with a physician, family, friend, etc., remotely. The patient care device can also display allergies, contraindications, drug interactions, etc. For example, FIG. 3 illustrates a pulse oximeter 301 with display 302. The pulse oximeter 301 includes pulse oximeter measurements of oxygen saturation 303 and respiration rate 307 with corresponding graphs 305 and 309. The pulse oximeter also includes buttons or controls 311 and microphone and/or speaker 314. The pulse oximeter also has sensor 313 attached to it. In an embodiment, such as shown in FIG. 4, the pulse oximeter displays advertisement or message 401 on the display 302. The message can include, for example, a soothing pictures or soothing sounds designed to relax the patient. Alternatively, the message can be a text, video and/or voice message from a doctor, nurse, other patient care giver, friend or family.

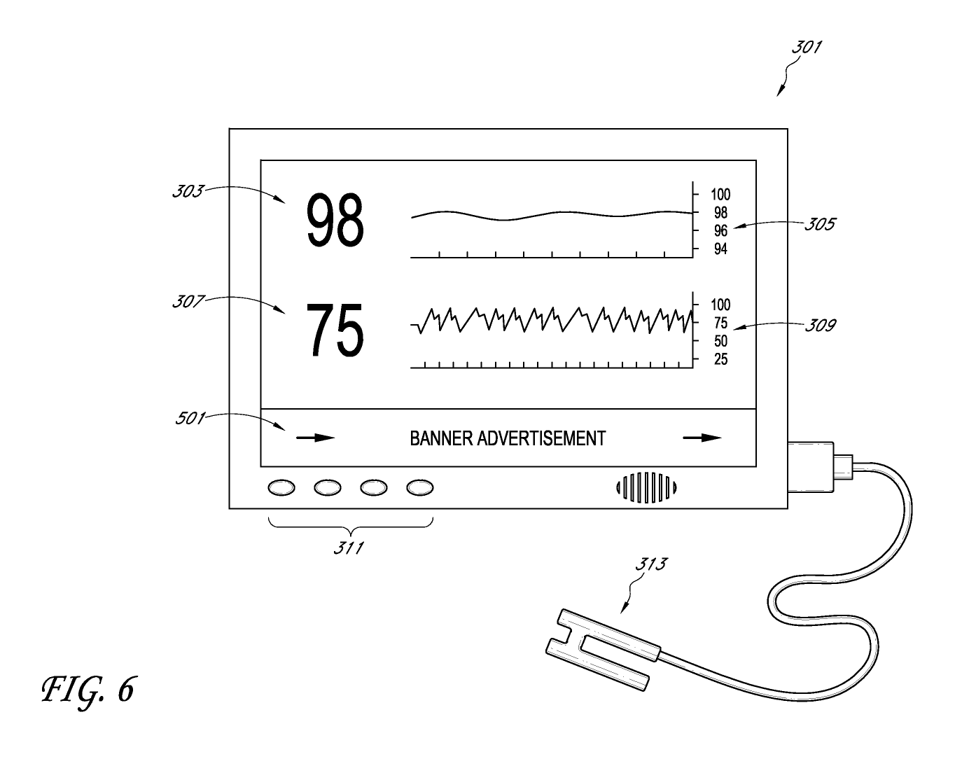

FIGS. 5 and 6 illustrate embodiments in which the message occupies only a portion of the screen while measurement information occupies another portion of the screen. This is advantageous to allow the display of measurement information while at the same time providing display of the message or advertisement. FIG. 5 illustrates a split screen arrangement with advertisement 401 occupying only a portion of the screen. FIG. 6 illustrates another arrangement in which a banner add or message is displayed across the bottom of the screen. As will be understood by a person of skill in the art from the present disclosure, any number of split screen arrangements can be used with the present disclosure and that FIGS. 5 and 6 are provided as an example only and are not intended to be limiting.

In an embodiment, the system determines when to display or play messages or advertisements on the patient care device so as to preserve the critical functionality of the system and the allow for unobstructed care of the patient. In an embodiment, messages and advertisements are only displayed when measurements are within normal ranges. In an embodiment, the system determines whether all readings for a given patient across all patient care devices are in a normal range before allowing messages. In an embodiment, some messages, such as messages from a physician are provided a higher priority and can be displayed even if readings are out of a normal range. In an embodiment, the system determines whether a care giver is present in the patient's room or area and prevents non-critical messages from being displayed. For example, a determination of who is present in a room may be made using RFID tags worn by care givers and/or patients. In an embodiment, patient's can initiate messages from the patient care device, for example, to pose a question to their physician.

In an embodiment, content, including messages and advertisements can be personalized or targeted at specific patients based information about the patient. For example, if the patient suffers from a particular illness, advertisements regarding non-generic drugs may be displayed. As another example, a newly admitted patient may be given a basic overview of the hospital or a tutorial of amenities of the hospital room.

In an embodiment, some or all of the patient care equipment, including patient monitors and other devices on the care facility network are provided with a configuration and/or management system. In an embodiment, the configuration and/or management system includes a configuration and/or management database associated with individual devices, groups of devices, devices associated with one or more domains, or all devices on the patient care network. In an embodiment, a configuration database is associated each patient care device and or device on the patient care network, including patient monitors. In an embodiment, a configuration database is associated with each measurement module of a patient monitor. The configuration and/or management database may be stored and/or operated on a server, a patient care device, or other computing or storage hardware. Additionally, the database may be made redundant across multiple servers, for purposes of reliability and fault tolerance.

In an embodiment, the configuration and/or management database is configured to allow authorized users to change, manage and/or update software, configurations, firmware, measurement parameters, licensing information, including licensing information related to measurement parameters, or the like. Configurations, can include, such as, for example, individual measurement capabilities, monitoring parameters, and adjustments to monitoring parameters such as averaging time, probe off settings, screen settings, alarm settings, and any other configurable settings. In an embodiment, the database can include, such as, for example, settings, firmware/software revisions, communications protocols, or any other configurable settings including consumable supported by the associated device(s). Consumables can include, such as, for example, sensors, cables or other disposable, resposable or reusable accessories used in conjunction with the device.

In an embodiment, the configuration database can be accessed remotely. In an embodiment, the configuration database can be accessed locally on each device or locally on the patient care facility network. In an embodiment, different levels of configuration access are provided. For example, an embodiment, a hospital technician is provided with security clearance to adjust alarm sensitivity, display features, or adjust measurement parameters. However, the hospital technician may be prevented from changing the type of parameter a patient care device is authorized to measure, whereas a remote sales representative is provided with security clearance to change which parameters a device is configured to measure. In an embodiment, devices are provided with a list of privileges and scope rules. In an embodiment, the list is provided at the factory and run time rules are provided to govern the management of the configuration information in the field.

In an embodiment, a management platform is provided. In an embodiment, the management platform can be hosted and/or managed by the manufacturer or an authorized party. In an embodiment, the management platform manages configuration settings, software and firmware updates, etc. for devices on the patient care facilities network, including patient monitors and other patient care devices. In an embodiment, the management platform provides a set of policies that map specific privileges, settings, roles and scope that are available. In an embodiment, the management platform provides a set of compliance and service reports that track the enforcement of the policy settings to the actual behavior of the system. For example, in an embodiment, when a hospital desires to upgrade a device to measure an additional parameter, the management platform can send instructions to a device's configuration database to allow for the addition parameter. Likewise, the management platform can assimilate information from various devices to confirm settings, such as the types of parameters a device is configured to measure and confirm that the device is actually measuring those parameters.

In an embodiment, patient care devices include a feature of automatic preference adjustment. Medical devices often have various preference options that affect the operation and output displays of the device. These preference options include, but are not limited to, display colors, font sizes, font styles, display brightness, measurement units (e.g. metric vs. UK), refresh rate, sampling rate, language, volume, preferred output devices, email addresses, telephone or pager numbers, and so forth. In an embodiment, preference options associated with a user are stored on a storage medium, and may be maintained in a database. The storage medium may be on the device itself, or it may be accessible to the device via a network or other means.

In order to provide automatic preference adjustment, the device may have means for detecting the presence of a person, such as a hospital staff member, in the proximity of the device. An RFID tag sensor is one means for detecting the presence of a person, but there are other equally functional means, such as a radio transmitter/receiver, a cell phone, a GPS tracker, a weight sensor, or the like. Once the device detects the presence of a person, the device may retrieve the stored preference options associated with the person. The device may then automatically update itself based on those stored preference options.

Additionally or alternatively, the device may notify other devices, so that the other devices may update themselves. This way, only one device needs to detect the person, but preferences can be updated across many devices, some of which may lack the ability to detect the presence of a person. The device may notify the other devices via an open architecture network, as described in this specification, by transmitting the stored preference options directly to the other devices, or it may instruct the other devices to retrieve the stored preference options. In one embodiment, only devices associated with the same domain as the initial device will have their preferences updated. Rules stored on the device or on other storage media accessible to the device may be used to determine the manner of transmitting the stored preference options to other devices, the devices that are to receive the options, the options to be propagated, and other relevant information.

Those of skill in the art will understand that information and signals can be represented using any of a variety of different technologies and techniques. For example, data, instructions, commands, information, signals, bits, symbols, and chips that can be referenced throughout the above description can be represented by voltages, currents, electromagnetic waves, magnetic fields or particles, optical fields or particles, or any combination thereof.

Those of skill will further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of the present invention.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, conventional processor, controller, microcontroller, state machine, etc. A processor can also be implemented as a combination of computing devices, e.g., a combination of a DSP and a microprocessor, a plurality of microprocessors, one or more microprocessors in conjunction with a DSP core, or any other such configuration. In addition, the term “processing” is a broad term meant to encompass several meanings including, for example, implementing program code, executing instructions, manipulating signals, filtering, performing arithmetic operations, and the like.

The steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, a DVD, or any other form of storage medium known in the art. A storage medium is coupled to the processor such that the processor can read information from, and write information to, the storage medium. In the alternative, the storage medium may be integral to the processor. The processor and the storage medium can reside in an ASIC. The ASIC can reside in a user terminal. In the alternative, the processor and the storage medium can reside as discrete components in a user terminal.

The modules can include, but are not limited to, any of the following: software or hardware components such as software object-oriented software components, class components and task components, processes, methods, functions, attributes, procedures, subroutines, segments of program code, drivers, firmware, microcode, circuitry, data, databases, data structures, tables, arrays, or variables.

In addition, although this invention has been disclosed in the context of a certain preferred embodiment, it will be understood by those skilled in the art that the present invention extends beyond the specifically disclosed embodiment to other alternative embodiments and/or uses of the invention and obvious modifications and equivalents thereof. In particular, while the present system and methods have been described in the context of a particularly preferred embodiment, the skilled artisan will appreciate, in view of the present disclosure, that certain advantages, features and aspects of the system, device, and method may be realized in a variety of other applications and software systems. Additionally, it is contemplated that various aspects and features of the invention described can be practiced separately, combined together, or substituted for one another, and that a variety of combination and subcombinations of the features and aspects can be made and still fall within the scope of the invention. Furthermore, the systems described above need not include all of the modules and functions described in the preferred embodiments. Thus, it is intended that the scope of the present invention herein disclosed should not be limited by the particular disclosed embodiment described above, but should be determined only by a fair reading of the claims that follow.

What is claimed is:

1. A patient monitoring system connected to an open architecture patient care network, the patient monitoring system comprising:
an electromagnetic wave emitter configured to generate electromagnetic radiation having a predefined wavelength;
an optical sensor configured to measure intensity of the electromagnetic radiation after attenuation of the waves through patient tissue and produce sensor data corresponding to said intensity; and
a virtual machine, executed on a processor, configured to receive sensor data from the optical sensor, transform the sensor data into a format compatible with an open architecture network, and transmit the transformed sensor data to a second device connected to the open architecture patient care network, the second device configured to perform a post-processing function on the transformed sensor data.

2. The patient monitoring system of claim 1, wherein the virtual machine is executed on a device housing the optical sensor.

3. The patient monitoring system of claim 1, wherein the virtual machine is executed on a device not housing the optical sensor.

4. The patient monitoring system of claim 1, wherein the post-processing function comprises displaying a graphical representation of the transformed sensor data on a display of the second device.

5. A system which integrates multiple devices in a patient care facility that operate on different platforms and with different specifications, the system comprising:
a plurality of patient monitors configured to operate using a first operating system and further configured to measure physiological measurements;
a plurality of virtual machines executing on the plurality of patient monitors and configured to translate operating parameters of the patient monitors for communication over a network; and
a server device configured to communicate with the plurality of virtual machines over the network, monitor the plurality of patient monitors, and respond to a fault detected in one of the plurality of patient monitors.

6. The system of claim 6, further comprising a physician operated device that is configured to receive the communications over the network and provide interactive management of the plurality of patient monitors, wherein the physician operated device includes a virtual machine for interpreting the communications.

7. The system of claim 6, wherein the physician operated device and at least one of the plurality of patient monitors are synchronized.

8. The system of claim 6, wherein the fault is selected from a group comprising: no communication with pulse oximeter, alarm silenced on pulse oximeter, instrument low battery (pulse oximeter), transmitter low battery, SpO₂ levels and alarms, high and low SpO₂, high and low PR, HbCO level and alarms, HbMET level and alarms, pulse rate and alarms, no sensor, sensor off patient, sensor error, low perfusion index, low signal quality, HbCO, HbMET, PI trend alarm, and desat index alarm.

9. The system of claim 6, wherein responding to the fault comprises: receiving a communication over the network indicative of the fault, identifying a rule associated with the fault, and responsively relocating a post-processing function of at least one of the plurality of patient monitors in accordance with the identified rule.

10. The system of claim 6, wherein at least one of the plurality of patient monitors is associated with a domain, and wherein the server device is associated with the domain.

11. The system of claim 10, wherein the at least one of the plurality of patient monitors is configured to perform a method comprising: connecting to the network; receiving data descriptive of other patient care devices associated with the domain; determining, based on a rule, whether a minimum set of patient care devices is present for operation; and generating an alert where the minimum set of patient care devices is not present.

12. A non-transient computer readable medium comprising instructions that, when executed on one or more processors, are configured to instantiate a virtual machine system connected to a patient monitoring device and an open architecture patient care network, the virtual machine system comprising:
a sensor management module configured to receive sensor data from a sensor of the patient monitoring device, the sensor data being in a proprietary format specific to the device, translate the sensor data into translated sensor data compatible with the open architecture patient care network, and transmit the translated sensor data over the network;
a fault monitoring module configured to test the patient monitoring device for faults and, upon detecting a fault, transmit data descriptive of the fault to one or more other devices on the network, the data being compatible with the open architecture patient care network; and
a preference manipulation module configured to receive an instruction over the network, the instruction formatted compatibly with the open architecture patient care network, and adjust one or more parameters of the patient monitoring device in accordance with the instruction.

13. The non-transient computer readable medium of claim 12, wherein the virtual machine communicates with the patient monitoring device via a network.

14. The non-transient computer readable medium of claim 12, wherein the virtual machine manages more than one patient monitoring device.

15. The non-transient computer readable medium of claim 12, wherein sensor data includes at least one type of data from a list of data types comprising: services related to sensor channels, channel errors, sensor off, sensor expired, calibration information, channel exceptions, measurement raw type, measurement limits, alarm levels, alarm priority, numerics including waveforms and measurement numbers, multiple channel i/o, sampling interval, display attributes, multiple levels of alarm thresholds, averaging, 3D alarms, system faults, configurations and settings, accounting, performance, security, authentication, integrity, privacy, interface connections, alarm engine activation, and status of alarms.

16. The non-transient computer readable medium of claim 12, wherein the virtual machine system further comprises a content management services module configured to push and pull externally originating content and display the externally originating content on a display of the patient monitoring device.

17. A system for using a patient monitor as a communication medium without disrupting the core functionality of the patient monitor, the system comprising:
a patient monitoring device including a display, the patient monitor configured to measure one or more physiological parameters of a patient;
a network in electrical communication with said patient monitor and configured to exchange communications with said patient monitor; and
a communications management module configured to manage communications presented on said patient monitoring display, the communications management module configured to determine, based on priority levels of a communication, when to display the communication on the display.

18. The system of claim 17, wherein the communications management module runs on the patient monitoring device.

19. The system of claim 17, wherein the communications are not displayed when monitored readings are outside of a normal range.

20. The system of claim 17, wherein the communications comprise one or more of an email, a text message, a voice message, a video message, a telephone call, a video conference, an advertisement, an information message, entertainment, and soothing content.

21. A method of monitoring patient care devices connected to an open architecture network, the method executed on one or more processors of a management system, comprising:
maintaining, on a non-transient computer-readable storage medium, a set of rules for responding to alerts raised by different types of patient care devices in a manner specific to each type of patient care device;
receiving information over the network identifying each of a plurality of patient care devices as being associated with a domain;
monitoring the network for information transmitted from any of the plurality of patient care devices indicative of an alert;
receiving information from an alerting patient care device among the plurality of patient care devices indicative of an alert originating from the alerting patient care device; and
responding to the alert in accordance with a portion of the set of rules, wherein responding to the alert comprises transmitting data to a receiver device via the network or a different network.

22. The method of claim 21, wherein the receiver device is a clinician end-user device configured to display information descriptive of the alert.

23. The method of claim 21, wherein responding to the alert comprises relocating a post-processing function of the alerting patient care device to a different patient care device.

24. The method of claim 21, wherein the set of rules includes rules for responding to a change in location of a patient care device.

25. The method of claim 21, further comprising: identifying the presence of a location-indicative tag, retrieving preference options associated with the location-indicative tag, and instructing one or more of the plurality of patient care devices to update displays based on the retrieved preference options.

26. A computer-implemented method of preventing a service interruption of a patient monitoring device having a sensor, the method comprising:
disabling a post-processing function being performed on the patient monitoring device;
enabling a substitute post-processing function on a second device; and
transmitting sensor data, originating from the sensor of the patient monitoring device, to the second device via a network, the sensor data being used as an input to the substitute post-processing function.

27. The method of claim 26, wherein the method is performed on a processor of the patient monitoring device.

28. The method of claim 26, wherein the method is performed on a processor of a server device, the server device being different from the patient monitoring device and the second device.

29. The method of claim 26, wherein the second device is a second patient monitoring device of a type different from the type of the patient monitoring device.

30. The method of claim 26, wherein the method is performed upon detecting an alarm raised by the patient monitoring device.
